(19) United States
(12) Patent Application Publication
(10) Pub. No.: US 2025/0130246 A1
(43) Pub. Date: Apr. 24, 2025

(54) METHOD OF DETECTING PROTEIN AGGREGATES

(71) Applicant: CAMBRIDGE ENTERPRISE LIMITED, Cambridge, Cambridgeshire (GB)

(72) Inventors: Aviad LEVIN, Cambridge, Cambridgeshire (GB); Tuomas KNOWLES, Cambridge, Cambridgeshire (GB)

(21) Appl. No.: 18/706,270

(22) PCT Filed: Nov. 1, 2022
(86) PCT No.: PCT/GB2022/052752

§ 371 Date: Apr. 30, 2024

(30) Foreign Application Priority Data
Nov. 1, 2021 (GB) 2115639.3

(51) Int. Cl. G01N 33/68; G01N 33/58

(52) U.S. Cl. CPC G01N 33/6896; G01N 33/582; G01N 2800/2821; G01N 2800/2835

(57) ABSTRACT

The disclosure relates to methods of investigating protein aggregation reactions, in particular methods for detecting aggregates of a protein that are capable of seeding further protein aggregation. The methods allow not only understanding of aggregation reactions, but also provide means for detecting whether a sample from an individual comprises aggregate seeds.

### FIELD OF THE INVENTION

The present invention broadly relates to methods of examining protein aggregation, in particular to methods of detecting aggregates of a protein in a sample that are capable of seeding further protein aggregation and amplification. The methods of the invention allow secondary aggregation and primary aggregation processes to be evaluated individually.

The work leading to this invention has received funding from the European Research Council under the European Union's Seventh Framework Programme (FP7/2007-2013)/ERC grant agreement No 337969.

### BACKGROUND OF THE INVENTION

Proteins are the fundamental physical building blocks of life, underpinning almost all of the molecular processes in biological systems. They are able to exert their biological activities typically by binding to other proteins to form functional complexes which act as the machinery of life. As such, the ability of proteins to recognise their partners in the highly crowded milieu of the cellular cytoplasm is central to biological function.

In certain cases, however, proteins escape these quality control mechanisms and form aberrant complexes. In particular, the formation of β-sheet rich clusters stabilised by inter-molecular hydrogen bonds, amyloid aggregates, has dramatic consequences for biological systems and has been implicated in the onset and development of neurodegenerative disorders. This phenomenon has inspired a sustained research effort to elucidate the basic physical principles which govern self-assembly and the nature of the structures that emerge as a result of protein aggregation, in contexts ranging from the development of artificial materials to understanding human disease. Protein misfolding and aggregation disorders, such as Alzheimer's disease (AD), Parkinson's disease (PD), and many other age-related neurodegenerative conditions, are associated with the formation of such aberrant supra-molecular clusters from normally monomeric proteins and peptides, such as Aβ and α-synuclein, resulting in the accumulation of pathological aggregates, which play a central role in the pathogenesis of these diseases (Dobson (1999), Spillantini et al (1997), Padrick et al (2002), Chiti et al (2002) and Lotharius et al (2002)).

The initial aggregates, formed through primary nucleation or the introduction of structures capable of seeding the reaction, can grow further through incorporation of monomers from the solution to form elongated fibrils (Knowles et al (2009)). The direct primary nucleation pathway is, in many cases, followed by secondary nucleation processes, which allow existing aggregates to self-replicate (Knowles et al (2009) and Cohen et al (2013)). As such, these structures, once formed, are often able to self-replicate in a prion-like manner, leading to highly complex dynamics in both time and space. This auto-catalytic chain reaction results in a positive feedback loop that leads to a rapid generation of new aggregates and an exponential increase in the amount of aggregated species, and is the cause of the typical sigmoidal aggregation curves with long lag times observed during in vitro aggregation experiments (Meisl et al (2014)).

The amplification of signals derived from aggregates is currently at the forefront of diagnostic requirements, as it allows the detection of a small number of aggregates capable of propagation in biological samples. As such, real-time quaking-induced conversion (RT-QuIC) and protein misfolding cylic amplification (PMCA) assays have recently been developed and provide signal amplification of aggregation prone proteins in solution, such as prions (Wilham et al (2010), Atarashi et al (2011) and McGuire et al (2012), αSyn aggregates (Shahnawaz et al (2017), Fairfoul et al (2016) and Groveman et al (2017) and other amyloids (Saijo et al (2017) and Salvadores et al (2014) at concentrations as small as one in a million. These approaches rely on the fragmentation of growing fibrils through shaking to allow their exponential aggregation, requiring 20-40 hr of incubation to reach a detectable seed concentration equivalent to 10⁻⁶-10⁻⁹ M of the monomer (Saijo et al (2019) and Han et al (2020)). While this approach allows the detection of aggregation-prone species in biological bulk samples, detecting and quantifying single aggregation prone species remain challenging.

Droplet-based microfluidics provides a key advancement in the direct analysis of protein aggregation at a single aggregate resolution. Specifically, the present inventors have previously developed and introduced to the field of protein aggregation digital approaches. In a digital measurement, a binary signal is recorded for the presence or absence of a specific biological entity, either through amplification or through direct measurements of individual species one by one. By contrast, in an analogue measurement, a continuous signal is recorded that informs on the overall state of the sample that contains many entities. Digital measurements provide fundamental advantages: since they are based on discrete counting, they are inherently calibration free, and since they sample the elementary constituents of a heterogeneous sample individually, they are able to deal with heterogeneity of complex matter at the ultimate resolution of single species.

The power of microfluidics has previously been leveraged to develop a single aggregate detection assay for the quantitative analysis of insulin aggregates (Pfammatter et al (2017)). However, this approach provides a similar detection limit to that of RT-QuIC and as it requires the generation of microdroplet arrays in glass capillaries, it does not allow for a facile and high-throughput method for the detection of aggregates.

### SUMMARY OF THE INVENTION

An aim of the present invention aim was to go beyond the state of the art and address the fundamental problems originating from studying heterogeneous biomolecular systems through the development and application of a new set of biophysical techniques to study aberrant protein-protein self-assembly on the level of single aggregates to resolve the fundamental mechanisms underlying their formation and activity at the single molecule level.

Accordingly, the present inventors have developed a method for the detection of single aggregates encapsulated within microfluidic droplets, relying on increasing the concentration of the monomeric precursor species inside the droplet. Through forming arrays of droplets containing aggregate seeds and soluble protein monomers, controlled evaporation of the droplet volume is achieved. While the approach of inducing the evaporation of droplets have been previously utilised for monitoring the primary nucleation of solids and liquid condensates (Kopp et al (2020) and Levin et al (2018) the method of the present invention allows for secondary processes to be detected and the propagation of seeds to be quantified. Thus, through employing this approach, the concentration of soluble monomers increases over time due to droplet shrinkage, thus allowing for a critical monomer concentration to be reached to allow for the detection of aggregate propagation within individual droplets. This approach benefits from a clear signal-to-noise ratio of aggregated droplets as compared with those devoid of seeds. Additionally, the approach allows for a detection limit of 10⁻¹² M seed concentration (monomer equivalent concentration), and a digital signal is achieved within 10⁻¹⁴ hours. The methods of the present invention will therefore reduce the current limit of detection and allow to investigate the ability of single aggregates to propagate in a rapid manner through minimizing the assay volume and thus open new opportunities for the diagnosis of a wide range of protein aggregation-based diseases.

The inventors have also developed a further method where fragmentation of aggregate seeds is induced and the formation of further aggregates are then detected. The fragmentation is induced via changing the shape of microdroplets (typically squeezing the microdroplets) in order to increase collision between the aggregate seeds and an agent present in the microdroplets, which agent is capable of fragmenting the seed upon collision. Such methods may either be used alone, or in combination with the above-described methods of increasing monomer chemical potential.

Accordingly, the present invention provides a method of detecting aggregates of a protein that are capable of seeding further protein aggregation, said method comprising:
(a) providing a preparation that (i) comprises, or is suspected of comprising, protein aggregate seeds of said protein and (ii) comprises monomers of said protein;
(b) generating microdroplets of the preparation of (a);
(b) increasing the monomer chemical potential within the droplets to a point where secondary aggregation processes take place, but not primary aggregation processes; and
(c) determining the presence or absence of aggregation within the droplets;
wherein the concentration of protein aggregate seed in the mixture of step (a) is such that the overwhelming majority of microdroplets of step (b) will each contain either one or zero seeds.

The invention also provides a method of detecting aggregates of a protein that are capable of seeding further protein aggregation, said method comprising:
(a) providing a preparation that (i) comprises, or is suspected of comprising, protein aggregate seeds of said protein, and (ii) comprises monomers of said protein;
(b) generating microdroplets of the preparation of (a);
(c) modifying the shape of the droplets;
(d) determining the presence or absence of aggregation within the droplets;
wherein the concentration of protein aggregate seed in the mixture of step (a) is such that the overwhelming majority of microdroplets of step (b) will each contain either one or zero seeds.

In particular, the provides a method of detecting aggregates of a protein that are capable of seeding further protein aggregation, said method comprising:
(a) providing a preparation that (i) comprises, or is suspected of comprising, protein aggregate seeds of said protein, (ii) comprises monomers of said protein and (iii) comprises an agent capable of fragmenting the aggregate seeds following a collision between the seeds and the agent;
(b) generating microdroplets of the preparation of (a);
(c) modifying the shape of the droplets to increase the chance of collisions between the aggregate seeds and the agent capable of fragmenting the aggregate seeds;
(d) determining the presence or absence of aggregation within the droplets;
wherein the concentration of protein aggregate seed in the mixture of step (a) is such that the overwhelming majority of microdroplets of step (b) will each contain either one or zero seeds.

### BRIEF DESCRIPTION OF THE FIGURES

FIG. 1 shows a schematic of microdroplet generation (top left) loaded with protein monomer and aggregates. Droplets are then arrayed within a PDMS microwell device where evaporation takes place (top right). Evaporation of droplet volume then leads to an increase of monomer concentration and their chemical potential to interact with protein seeds present, thus amplifying the fluorescent (ThT) signal correlated with amyloid propagation (bottom).

FIG. 2 shows a limit of detection comparison in constant vs. reduced droplet volumes. A. ThT signal propagation in seeded microdroplets at concentration of 20 nM, 2 nM and 2 pM without evaporation, incubated at 37° C. for 1200 min. B. Seed amplification at 200 pM concentration following evaporation. Top, brightfield image of droplets immobilised in arrays. Middle, Fluorescent image of ThT signal of propagons following 830 min incubation at 37° C. Bottom, merged brightfield (0 min) and fluorescent image (830 min). Scale bar represents 50 μm.

FIG. 3 shows ThT positive vs. negative microdroplet fraction at decreasing seed concentrations. Brightfield (top) and Fluorescent ThT (bottom) images were acquired at time 0 and following 920 min of incubation at 37° C. As seed concentration is decreased so does the fraction of ThT positive droplets. Seed only and ThT only fluorescent images were further acquired as controls. Scale bar represents 50 μm.

FIG. 4 shows quantification of droplet diameter reduction and limit of detection. A. Initial droplet diameter averaged and the diameter of ThT positive and negative droplets following incubation of droplets containing 200 pM seeds. The line represents the corresponding volume reduction of positive (10 fold) and negative (22 fold) droplets. n=350 droplets. B. Fraction of ThT positive droplets at seed concentration ranging from 2 μM-200 fM. through serial dilution of the sample the fraction of positive signal is dramatically reduces to ˜3%. n=1600 droplets

FIG. 5 shows a “shearing” method of the invention where microdroplets are formed containing monomers, aggregate seeds and a fragmentation agent (beads). The microdroplets are passed through a narrow channel, which deforms the droplets and compresses them. This compression allows the beads to collide with the seeds and induce fragmentation. Further fibrillary fragments then grow though the addition of monomer. This yields an increase in reporter signal (e.g. ThT).

FIG. 6 shows results from a digital detection method, where fluorescent dye is added to the droplet in order to image the droplet boundaries.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise.

All publications, patents and patent applications cited herein, whether supra or infra, are hereby incorporated by reference in their entirety.

The invention provides a method of detecting aggregates of a protein that are capable of seeding further protein aggregation. The method comprises providing a preparation that (i) comprises, or is suspected of comprising, protein aggregate seeds of said protein and (ii) comprises monomers of said protein. The method then comprises generating microdroplets of the preparation of and increasing the monomer chemical potential within the droplets to a point where secondary aggregation processes takes place, but not primary aggregation processes. Finally, the method comprises determining the presence or absence of aggregation (i.e. the formation of new aggregates and/or the growth of existing aggregates) within the droplets.

### Providing a Preparation That Comprises or is Suspected of Comprising Protein Aggregate Seeds and Protein Monomers

Protein aggregation is a biological phenomenon where proteins accumulate either inside or outside of cells. Various mechanisms of aggregation exist. However, aggregation typically begins with a mis-folded (or partially unfolded) protein. Mis-folding may occur for a number of reasons, including mutations in the amino acid sequence of the protein or as a result of environmental stress, such as pH, temperature or oxidative stress. Changes in the mis-folded protein structure then promote the formation of aggregates. For example, the exposure of hydrophobic residues within the protein may initiate an interaction with hydrophobic residues in a second protein monomer.

As discussed further below, the present invention can be applied to any protein which forms aggregates. However, the invention typically relates to amyloidogenic proteins.

Amyloids are aggregates of proteins typically characterised by a fibrillary morphology of 7-13 nm in diameter and a β-sheet secondary structure (known as cross-β sheet). Amyloid fibrils may be deposited in the form of insoluble extracellular plaques or intracellular inclusions. As discussed further below, the formation of amyloid fibrils is associated with a range of diseases, including Alzheimer's and Parkinson's.

The formation of amyloid fibrils involves both primary processes, and secondary processes (see Meisl et al (2014), Törnquist et al (2018) and Gaspar et al (2017)). In primary nucleation, soluble protein monomers interact to form oligomers, which grow and convert into proto-fibrils then fibrils. Primary nucleation is therefore a reaction involving monomers, without contributions from already formed aggregates. Secondary processes may then be either monomer-dependent or monomer-independent. Monomer independent secondary processes generate new aggregates at a rate that solely depends on the level of existing aggregates. Any example of such a monomer independent process is fragmentation, where an existing fibril fragments into two or more new fibrils. Monomer-dependent secondary processes then create new aggregates at a rate that depends on both the concentration of monomeric protein and existing aggregates. Surface-catalysed nucleation (secondary nucleation) is a monomer-dependent secondary process, where protein monomers interact with fibrils which serve as catalytic sites for the generation of new toxic oligomeric species. These oligomers can grow and convert into additional fibrils, thus resulting in a cycle of promotion of formation of toxic species.

Protein aggregation, and the formation of amyloid fibrils, is typically represented by a time-dependent sigmoidal curve. Such curves typically comprise a lag phase, an elongation phase and a plateau. The initial lag phase of the curve typically represents nucleation reactions (the formation or oligomers from monomers) and elongation typically represents the accumulation of oligomeric nuclei, which act as seeds for the formation proto-fibrils. During saturation (the plateau), mature fibrils are then produced from protofibrils. Addition of fibrils (or other functional seeds) at the start of the reaction allows elongation to proceed without the need for primary nucleation, thus removing the lag phase.

As mentioned above, in the invention the protein may be any protein which forms aggregates. The protein is typically an aggregation prone-protein. An aggregation-prone protein is a protein that has a propensity to self-aggregate. Aggregation-prone proteins may non-amyloidogenic. However, preferably, the protein is an amyloidogenic protein, i.e. a protein that forms amyloid fibrils.

Examples of amyloidogenic proteins include Aβ42, α-synuclein, tau, huntingtin, atrophin-1, ataxin (1,2,3,6,7, 8 12,17), amylin, prion protein, (pro)calcitonin, atrial natriuretic factor, apoliprotein AI, apoliprotein AII, apoliprotein AIV, serum amyloid, medin, (apo) serum AA, prolactin, transthyretin, lysozyme, β-2 microglobulin, fibrinogen α chain, gelsolin, keratopthelin, β-amyloid, cystatin, ABriPP immunoglobulin light chain AL, immunoglobulin heavy chain, S-IBM, islet amyloid polypeptide, insulin, lactadherin, lactoferrin, tbn, leukocyte chemotactic factor-2, AbriPP, ADanPP, lung surfactant protein, galectin 7, corneodesmosin, lactadherin, kerato-epithelium, odontogenic ameloblast-associated protein, semenogelin 1 and enfurvitide. Any of these proteins may be utilised in the present invention. Preferably, the protein is α-synuclein, tau or amyloid β (e.g. Aβ42), most preferably α-synuclein.

As set out above, the method of the invention relates to the detection of aggregates of a protein that are capable of seeding further protein aggregation. Such aggregates are known as protein aggregate seeds. In other words, the invention relates to investigating secondary processes.

In the invention, secondary processes refers to any means by which fibrils can propagate. Such secondary processes include monomer-dependent processes (secondary nucleation) and monomer-independent secondary processes (fragmentation), as well as growth of fibrils (elongation).

The method may further comprise an analysis of primary processes (primary nucleation). However, one the key features of the invention is the ability to analyse secondary processes independently from primary.

The method of the invention involves providing a preparation that comprises, or is suspected of comprising, protein aggregate seeds and comprises monomers of the protein. As discussed below, the preparation is typically an aqueous preparation. The protein aggregate seeds are any aggregate, but are typically fibrils, proto-fibrils or other functional seeds capable of starting an aggregation reaction (or capable of being elongated). The protein aggregate seeds and the monomers are derived from the same protein. For example, the method may involve the use of α-synuclein monomers and α-synuclein fibrils.

The preparation is typically prepared by mixing a sample comprising, or suspected of comprising, protein aggregate seeds with a different preparation comprising the protein monomers.

The sample comprising or suspected of comprising the protein aggregate seeds may be derived from a biological sample from an individual. “Individual” is used interchangeably herein with “patient” and “subject”. The individual may be any animal, but is typically a mammal. The individual is preferably human. However, the individual could also be a non-human mammal, such as a non-human primate, or a murine, bovine, equine, canine, ovine, or feline mammal.

The sample may be any appropriate sample type, such as any liquid, cell, or tissue obtained from an individual. In some aspects, the sample is blood, serum, plasma, cerebrospinal fluid (CFS), saliva, urine, or a tissue biopsy.

The individual may be diagnosed with, or is suspected of having, a disease associated with protein aggregation. For example, the individual may be diagnosed with, or suspected of having, Alzheimer's disease, Parkinson's disease, Huntington's disease, diabetes type 2, atrial amyloidosis, prion diseases (e.g. spongiform encephalopathies), primary systemic amyloidosis, senile systemic amyloidosis, haemodialysis-related amyloidosis, hereditary nonneuropathic systemic amyloidosis, injection-localized amyloidosis, secondary systemic amyloidosis, hereditary cerebral amyloid angiopathy or familial amyloidosis. The individual may also be diagnosed with, or suspected of having, any other synucleinopathy, which generally refers to neurodegenerative diseases characterised by the abnormal accumulation of aggregates of alpha-synuclein protein in neurons, nerve fibres or glial cells. There are three main types of synucleinopathy: Parkinson's disease (PD), dementia with Lewy bodies (DLB), and multiple system atrophy (MSA). Similarly, the individual may also be diagnosed with, or suspected of having, any tauopathy, which generally refers to neurodegenerative diseases involving the aggregation of tau protein into neurofibrillary or gliofibrillary tangles in the brain. Examples of tauopathies include primary age-related tauopathy dementia, chronic traumatic encephalopathy, progressive supranuclear palsy, corticobasal degeneration, frontotemporal dementia and parkinsonism linked to chromosome 17, lytico-bodig discase, ganglioglioma and gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, Pick's disease and corticobasal degeneration. Alzheimer's disease may also be considered a secondary tauopathy.

Individuals may present with any appropriate symptoms for the disease in question and/or may be diagnosed using any techniques known in the art.

For example, symptoms of Parkinson's disease include involuntary shaking, slow movement and stiff and inflexible muscles. Parkinson's is typically diagnosed based on an individual's symptoms, medical history and physical examination.

Early symptoms of Alzheimer's disease include memory loss, forgetfulness, lack of judgement and becoming more hesitant to try new things. Middle-stage symptoms include worsening memory problems, increased confusion and disorientation, problems with speech, changes in mood, disturbed speech, hallucinations and difficulties in performing spatial tasks. Late-stage symptoms include increasing severity of the earlier symptoms, difficulty in swallowing and eating, weight loss, loss of speech and significant memory problems. Diagnosis of Alzheimer's disease may involve physical examination, reviewing symptoms, blood and urine analysis, neurological examination, cognitive status testing and brain imaging (e.g. MI or CT).

Symptoms of Huntington's disease include difficulty concentrating and memory lapses, depression, clumsiness, involuntary jerking of the limbs, mood swings, difficult moving and problems swallowing, taking and breathing. Diagnosis often involves genetic testing.

In other instances, an individual may be asymptomatic. The individual may be asymptomatic but have a family history of the particular condition. The individual may also have a genetic pre-disposition for the condition.

The sample type may be any appropriate sample type for the disease in question. Examples of sample types are set out above. For example, the sample may be a serum or cerebrospinal fluid sample. Such samples may be obtained from the subject and treated/prepared using methods known in the art.

The sample comprising, or suspected of comprising, the seeds is typically diluted (in some instances, serial dilutions are prepared—see below). As is also discussed further below, samples are mixed with a separate preparation comprising the protein monomers.

Samples comprising, or suspected of comprising, aggregate seeds may be treated in any appropriate way prior to mixing with the preparation comprising the protein monomers (and/or the reagent which is capable of detecting the formation of aggregates; see below). For instance, as described in the examples, the sample may be sonicated (e.g. in a bath sonicator) in order to disperse any lumps of fibrils.

The buffer and pH for the sample comprising, or suspected of comprising, the seeds may be any appropriate buffer/pH depending on the protein in question. In some instances, the buffer may be phosphate buffered saline (PBS) or 2-(N-morpholino)ethanesulfonic acid (MES) buffer. In some preferred aspects of the invention, the buffer is PBS or MES and the protein is α-synuclein.

When the protein is α-synuclein, the pH is also typically acidic, for example between pH 4 and pH 6.5, between pH 5 and pH 6.5, or between pH 6 and 6.5. The pH could also be between pH 4 and pH 6 or between pH 5 and pH 6. For α-synuclein secondary processes are typically more evident in acidic pH. The pH could, however, also be approximately neutral (pH 6.5-7.5).

In addition to the protein aggregate seeds and the protein monomers, the preparation used to form microdroplets may further comprises a reagent which is capable of detecting the formation of aggregates. The reagent may be any small molecule or biological agent which is capable of distinguishing aggregates from, for example, protein monomers.

The reagent may emit a characteristic signal when in the presence of protein aggregates, such as when in the presence of amyloid fibres. The reagent may emit its characteristic signal when in the presence of beta-sheet rich structures.

The reagent may be an aggregation specific dye, in particular an aggregation specific small molecule dye. The reagent may be an aggregation specific fluorophore, or an aggregation specific non-fluorescent dye. A fluorophore is compound that can absorb light energy of a specific wavelength and re-emit light at a longer wavelength. The reagent may for example be a fluorophore or non-fluorescent dye that binds to beta sheet secondary structure and is therefore capable of detecting amyloid fibres. Such fluorophores may exhibit high fluorescence (and/or a shift in emission spectrum) when bound to beta-sheet rich structures.

Examples of fluorophores that are used to detect amyloid fibres are thioflavin T (ThT), thioflavin S (ThS) and thioflavin X (ThX). Thioflavin T emits weak fluorescence at around 427 nm when not bound to amyloid fibres. However, when bound to amyloid fibres and excited at 450 nm, ThT produces a strong fluorescence signal at approximately 482 nm. ThT has traditionally been the gold standard for the detection of amyloid fibres.

Thioflavin S is a homogenous mixture of compounds that results from the methylation of dehydrothiotoluidine with sulfonic acid. Like thioflavin T it binds to amyloid fibrils but not monomers and gives a distinct increase in fluorescence emission. However, unlike thioflavin T, it does not produce a characteristic shift in the excitation or emission spectra

Thioflavin X is then a next-generation probe, which has increased brightness and binding affinity compared with ThT.

An example of an aggregation specific non-fluorescent dye is Congo red. When used to stain amyloid fibres, Congo red has apple-green birefringence seen under crossed polarized light.

Other means for identifying amyloid fibres include metachromatic stains, such as crystal violet.

The reagent may also be an agent that is capable of binding to beta-sheet rich structures, such as amyloid fibres, and then binding of the agent is detected using appropriate means. The reagent may be conjugated to a further agent which allows for its detection. For example, the reagent may be coupled to a non-fluorescent label or fluorescent label. The reagent may in some instances be an antibody that selectively binds to beta sheet-rich structures such as amyloid fibres. The antibody should not bind, or should only minimally bind, protein monomers. In other words, immune-detection methods may be utilised in the invention. In such methods, a secondary antibody (e.g. a labelled secondary antibody) could be utilised to detect binding of the primary antibody.

Methods of the invention may comprise a step of mixing a sample that comprises, or is suspected of comprising, the protein aggregate seeds with the protein monomers to form the preparation of the invention. As discussed above, in some instances the sample is derived from an individual.

Methods of the invention may also comprise mixing the reagent which is capable of detecting the formation of aggregates with a preparation that comprises, or is suspected of comprising the protein aggregate seeds and monomers. In some alternative instances, the reagent may be mixed (a) with a preparation comprising the protein monomers or (b) with the sample comprising or suspected or comprising the protein aggregate seeds. In scenario (a) the monomer/reagent preparation is then mixed with the sample comprising or suspected or comprising the protein aggregate seeds. In scenario (b), the seeds/reagent preparation is then mixed with a preparation comprising the protein monomers. Mixing may occur separate from the microfluidics apparatus discussed below, or the apparatus may be configured to carry out these mixing steps.

The reagent which is capable of determining the presence of aggregates may be present at any appropriate concentration. The reagent should be present at a high enough concentration that it will be able to interact with aggregates present and provide a signal, but not so high that it results in inaccurate results (e.g. due to precipitation out of solution). Such amounts could readily be determined by the skilled person.

For example, the reagent may be ThT which is present at a concentration of 500 nM-15 μM, 500 nM-10 μM or 500 nM-5 μM. In some instances, the ThT could be present at a concentration of 1 μM-15 μM, 1-10 μM or 1-5 μM. Preferably, the ThT is present at a concentration of 1-10 μM. In some instances, the ThT may be present at a concentration of about 2 μM. These figures refer to the concentration in the preparation comprising both the protein aggregate seeds and the monomers (i.e. in the preparation that is used to generate the microdroplets). These figures are especially applicable to initial average droplet diameters of 75 to 200 μm, monomer concentrations of between 1-100 μM (optionally 10-50 μM, 20-30 μM or about 25 μM) and a concentration of seeds of between 200 fM and 2 nM monomer equivalent (see below in relation to these monomer and seed concentrations). As set out above, the protein is preferably α-synuclein.

With regards to protein monomers, these may also be prepared in an appropriate buffer and at an appropriate pH. For example, the buffer may be PBS or MES. In some preferred aspects of the invention, the buffer is PBS or MES and the protein is α-synuclein. As set out above, when the protein is α-synuclein the pH is preferably acidic as secondary processes are typically observed to occur for α-synuclein at acidic pH. However, the pH could be approximately neutral.

An appropriate concentration of protein monomers (in the preparation used to form the microdroplets) may be selected based on the known or predicted concentration of seeds in the preparation used to form the droplets. The concentration of monomers is in general higher than the concentration of seeds. In some instances, the concentration of monomers may be at least 1,000 times, at least 10,000, at least 100,000, at least 1,000,000 times or at least 10,000,000 times higher than the concentration of seeds. For example, the concentration of monomers could be between 1,000 and 1,000,000 times higher than the concentration of seeds, such as between 10,000 times higher and 1,000,000 times higher. In certain aspects, the concentration of seeds in the preparation used to form droplets could be in the pM or nM range and the concentration of monomers may be in the μM range. As discussed further below, seed concentrations are typically provided as a monomer equivalent.

In some instances the concentration of monomers in the preparation used to form the droplets may be 1-100 μM. In some instances, the concentration may be 10-50 μM. optionally 20-30 μM. In some instances the concentration may be about 25 μM. These figures are generally applicable across the invention, but may in particular be applied to α-synuclein where the seed concentration is between 200 fM and 2 nM (or intermediate ranges—see below).

Protein concentrations may be determined using routine techniques known in the art, including determination of the absorbance at 280 nm.

With regards to the concentration of aggregate seeds, the concentration of the seeds in the preparation used to form the microdroplets should be such that the overwhelming majority of microdroplets will each contain either one or zero seeds. Typically, at least 90% of the microdroplets should contain either one or zero seeds. The proportion of microdroplets containing more than one seed should be 10% or less. In some instances, at least 95% of the microdroplets should contain either one or zero seeds and the proportion of microdroplets containing more than one seed should be 5% or less. Such proportions mean that the method allows for the detection of secondary aggregation processes, with only a low occurrence of false positive results.

Having the overwhelming majority of microdroplets containing either one or zero seeds may be achieved by diluting the original sample comprising or suspected of comprising the protein aggregate seeds. In some instances, serial dilutions may be used in order to achieve the desired proportion of microdroplets containing either one or zero seeds. For example, a serial dilution scheme could be a series of 10-fold dilutions of the original sample or seed stock solution (such as shown in the examples below).

The overall concentration of species or probability of droplets containing one or zero seeds can be related to the number of times amplification was detected through simple Poisson statistics.

Seed concentrations are typically determined as a monomer equivalent. In order to determine the monomer equivalent, seeds are dissembled into protein monomers and then the monomer concentration determined (for example using the absorbance at 280 nm). Any appropriate conditions may be used for dissembling the aggregates, such a treatment with a denaturing agent. Guanidinium chloride is an example of a denaturing agent that may be used in the determination of monomer equivalent concentrations.

The concentration of seeds in the preparation used to form the microdroplets (i.e. the concentration of seeds in the droplets at the start of the experiment) is typically between 200 fM and 2 nM monomer equivalent. The concentration of seeds may be between 2 pM and 2 nM monomer equivalent, or between 20 pM and 2 nM monomer equivalent. The concentration of seeds may also be between and between 200 fM and 1 nM monomer equivalent, or between 2 pM and 1 nM monomer equivalent, or between 20 pM and 1 nM monomer equivalent. These values may generally be applied across the invention, but are typically applicable to α-synuclein (and in combination with the monomer and/or ThT concentrations discussed above).

The buffer of the preparation used to form the microdroplets may be any appropriate buffer. For example, the buffer could be PBS or MES (in some instances the buffer could be PBS or MES and the protein is α-synuclein).

The pH could be between 4 and 9. In some instances the pH may be approximately neutral (e.g. pH 6.5-7.5). In other instances, the pH may be acidic (pH 4-6.5) or alkali (pH 7.5-9). An appropriate pH may be determined based on known understandings of protein aggregation reaction conditions.

For example, when the protein is α-synuclein, the pH is typically acidic, for example between pH 4 and pH 6.5, between pH 5 and pH 6.5, or between pH 6 and 6.5. The pH could also be between pH 4 and pH 6 or between pH 5 and pH 6. For α-synuclein secondary processes are typically more evident in acidic pH. The pH could also be approximately neutral (pH 6.5-7.5).

### Generating Microdroplets

As set out above, the method of the invention comprises generating microdroplets from the preparation. Microdroplets may be generated using any means known in the art. Microdroplets are in particular generated using a microfluidics device. The microdroplets are typically generated by contacting the preparation comprising (or suspected of comprising) the protein seed aggregates and protein monomers (and, where appropriate, the reagent capable of detecting aggregation) (the dispersed phase) with a carrier fluid (the continuous phase). The carrier fluid is typically immiscible with the preparation comprising (or suspected of comprising) the protein seed aggregates and protein monomers (and, where appropriate, the reagent capable of detecting aggregation). For example, the preparation may be an aqueous preparation. The aqueous preparation may be buffered as appropriate depending on the protein in question (see above). The carrier fluid may then be, for example, oil. The microdroplets may therefore be water-in-oil droplets.

Oils for the preparation of water-in-oil microdroplets would be well known to the person skilled in the art. For example, the oil could be a fluorinated oil (such as Fluorinert™ FC-40 oil utilised in the examples below). Several fluorinated oils are available that have varying solubilities in water. The oil phase may comprise additional surfactants used to influence the interfacial tension between the dispersed and continuous phases. For example, the surfactant may be a fluorosurfactant with a fluorinated tail.

Triblock copolymer surfactants containing two perfluoropolyether (PFPE) tails and a polyethylene glycol (PEG) block head group are examples of fluorosurfactants with good biocompatibility that are typically used in such droplet generation. Other possible surfactants include fluorinated linear polyglycerols, which can be functionalised on their side-chains. Examples of appropriate surfactants are described in Wagner et al (2016).

Appropriate concentrations of surfactant would be known to the skilled person. The example, as shown in the examples, droplets may be formed using FC-40 oil containing 4% (v/v) fluorosurfactant.

Droplets may be formed using any appropriate method, but are typically formed using microfluidic techniques. In order for droplet formation to occur, the two immiscible phases are typically contacted with one another. Droplets may be formed either actively or passively. Active droplet formation requires external energy input, such as electrical, magnetic or centrifugal forces. Passive droplet formation may then use a standard technique, such as cross-flowing droplet formation, flow focusing droplet formation or co-flowing droplet formation. In cross-flowing droplet formation the continuous and aqueous phases run at an angle to each other, with the dispersed phase extending into the continuous phase (for example the channels containing the phases may be perpendicular to each other with the dispersed phase then intersecting the continuous phase, although other arrangements are possible). As would be understood by a person skilled in the art, the flow rates of the dispersed and continuous phases may be adjusted as necessary. Flow focusing then involves the dispersed phase flowing to meet the continuous phase (typically at an angle) then undergoing a constraint that creates a droplet. The constraint may be a narrowing of the channel. Once again, flow rates may be adjusted as would be understood by a person skilled in the art. Finally, in co-flowing the dispersed phase is enclosed inside a continuous phase channel. At the end of the dispersed phase channel, the fluid is stretched until it breaks from shear forces and forms droplets either by dripping or jetting.

Droplets are typically formed at room temperature.

Microfluidics devices for the formation of droplets are known in the art, and are discussed further below. The microfluidics device, including the channels for droplet formation, is typically made from polydimethylsiloxane (PDMS) and may be produced using soft lithography techniques.

The droplets are typically generated at an average (mean) size (diameter) of 10-200 μm. In some instances, droplets may have an average diameter of 10-150 μm, 10-100 μm, or 10-50 μm. In some instances, the average diameter could be 20-200 μm, 20-150 μm, 20-100 μm or 20-50 μm. In other instances, the average diameter could be 50-200 μm, 50-150 μm or 50-100 μm. The average diameter could also be about 10 μm, about 20 μm, about 50 μm, about 75 μm, about 100 μm, about 150 μm or about 200 μm.

More typically, the average diameter is 75-200 μm, 100-200 μm, 75-150 μm or 100-150 μm. The average diameter may also be about 100 μm (for example 100 μm+25 μm).

The average diameter of the droplets may depend on other experimental conditions. for example, the protein being analysed and the concentration of seeds. In some instances, for seed concentrations of between 200 fM and 10 pM monomer equivalent, optionally between 200 fM and 5 pM monomer equivalent or between 200 fM and 2 pM monomer equivalent the average droplet diameter is 145 μm to 200 μm (optionally 150±25 μm). In other instances, for seed concentrations of between 200 fM and 10 PM monomer equivalent, optionally between 200 fM and 5 pM monomer equivalent or between 200 fM and 2 pM monomer equivalent the average droplet diameter is 120 μm to 150 μm (optionally 150±25 μm). Once again, these figures are generally applicable across the invention but typically apply to α-synuclein.

The size of the droplets may be controlled using means known in the art, including channel geometry, channel aspect ratio and flow rates. Droplet size may be determined using optical techniques, such as by capturing images of the droplets. Droplet size may also be determined by using a directed light source (such as a laser or LED) or by electrical techniques, where a droplet passing over contacts provides an electrical signal.

Once the droplets have been formed, the droplets may transported to a secondary array device. In some instances, droplets are arrayed onto a static surface. For example, droplets may be arrayed into a microwell device, such as a microfluidic chip. The microwell device is typically made of polydimethylsiloxane (PDMS). Each microwell may contain a single microdroplet, or may contain multiple microdroplets. The size of the microwells should be appropriate for the size of droplets being analysed. In some instances, microwells may have a diameter of 50-250 μm (for an average droplet diameter of 100 μm), optionally about 120 μm. Following immobilisation of the droplets, additional oil solution may be injected into the device in order to remove any droplets not trapped within the microwells.

Microdroplets may also be contained within channels within the microfluidics device. The channels may also be made from PDMS.

In some instances, individual droplets may be analysed on the static surface, or may be dynamically analysed by being flowed by a detector. Such methods are described further below.

Droplets may be transported to the secondary array device via tubing. In some instances, tubing may be made from polytetrafluoroethylene.

### Increasing the Monomer Chemical Potential Within the Droplets to a Point Where Secondary Nucleation Takes Place, But Not Primary Nucleation

The next step in the method involves increasing the monomer chemical potential to a point where secondary processes take place, but not primary. As discussed above, secondary processes may then be either monomer-dependent (e.g. surface catalysed secondary nucleation) or monomer-independent (fragmentation of existing fibrils). The invention is primarily intended for analysing such monomer dependent processes, although monomer independent processes can also be examined.

Chemical potential of a species in a mixture is defined as the rate of change of free energy of a thermodynamic system with respect to the change in the number of atoms or molecules of the species that are added to the system. In the present instance, as discussed below, a simple change in concentration is encompassed by the reference to a change in the chemical potential.

The present inventors have found that increasing the monomer chemical potential within the microdroplets (i.e. increasing the concentration of the protein monomers) initially promotes the occurrence of secondary processes. Further increasing the monomer chemical potential may then promote the occurrence of primary processes. The methods of the invention therefore involve increasing the monomer chemical potential to a point where secondary processed take place, but not primary. At this point, formation of aggregates within the droplets is analysed. After the analysis, the monomer chemical potential may though be further increased to the point where primary processes take place, and further analysis conducted.

In order for secondary processes to take place, but not primary, the monomer chemical potential is typically increased 5-fold to 100-fold. The monomer chemical potential may for example be increased 5- to 15-fold, 5- to 20-fold, 5- to 30-fold, 5- to 40-fold, 5- to 50-fold, 5- to 60-fold, 5- to 70-fold, 5- to 80-fold or 5- to 90-fold. The monomer chemical potential may for example be increased 10- to 20-fold, 10- to 30-fold, 10- to 40-fold, 10- to 50-fold, 10- to 60-fold, 10- to 70-fold, 10- to 80-fold, 10- to 90-fold or 10-100-fold. The monomer chemical potential could also be increased 20- to 30-fold, 20- to 40-fold, 20- to 50-fold, 20- to 60-fold, 20- to 70-fold, 20- to 80-fold, 20- to 90-fold or 20- to 100-fold. In some instances, the monomer chemical potential could be increased 30- to 40-fold, 30- to 50-fold, 30- to 60-fold, 30- to 70-fold, 30- to 80-fold, 30- to 90-fold, or 30- to 100-fold. In some instances, the monomer chemical potential could be increased 40- to 50-fold, 40- to 60-fold, 40- to 70-fold, 40- to 80-fold, 40- to 90-fold, or 40- to 100-fold. In some instances, the monomer chemical potential could be increased 50- to 60-fold, 50- to 70-fold, 50- to 80-fold, 50- to 90-fold, or 50- to 100-fold. In some instances, the monomer chemical potential could be increased 60- to 70-fold, 60- to 80-fold, 60- to 90-fold, or 60- to 100-fold. In some instances, the monomer chemical potential could be increased 70- to 80-fold, 70- to 90-fold, or 70- to 100-fold. In some instances, the monomer chemical potential could be increased 80- to 90-fold, or 80- to 100-fold. In some instances, the monomer chemical potential could be increased 90- to 100-fold.

Monomer chemical potential could also be increased by at least 5-fold, at least 10-fold, at least 20-fold, as least 3-fold, at least 40-fold, at least 50-fold, at least 60-fold, at least 70-fold, at least 80-fold, at least 90-fold or at least 100-fold.

In some instances, the protein is α-synuclein and the monomer chemical potential is increased 5- to 20-fold.

In the invention, the monomer chemical potential may be increased by evaporation. In some instances, evaporation could be conducted at room temperature (e.g. 15-25° C.). Evaporation does, however, typically involve incubating the droplets at a desired temperature until the required monomer chemical potential, or reduction in droplet size (diameter), is achieved. The use of higher temperatures in general increases the evaporation rate.

The temperature used for evaporation could be about 37° C. (for example 37° C.±2° C.). The temperature could be also be, for example, 30-40° C., 30-50° C., 30-60° C., 40-50° C., 40-60° C. or 50-60° C. In some instances, the temperature could be 35-45° C. An appropriate temperature may be chosen in view of factors such as the droplet size, the array device and the aqueous solution within the droplets.

The means for incubating the droplets at the desired temperature can be any appropriate means known in the art, and may depend on the secondary array device. For example, when the droplets have been arrayed into microwells a heated microscope stage may be used for the incubation (see the examples below).

The time over which evaporation is conducted will in part depend on the temperature, and on other factors such as the volume of the droplet and the aqueous solution within the droplets (e.g. the concentration of components in the aqueous solution). For example, with higher temperatures shorter time periods could be used in order to achieve the required reduction in droplet size.

In some instances, evaporation may take place for periods of up to 24 hours, or periods of up to 20 hours, or up to 16 hours. Preferably, evaporation may take place over periods of 9-16 hours, 10-14 hours or 9-12 hours. Such incubation periods are in particular appropriate for droplets with average diameters in the range of 75-200 μm (or 75-150 μm, or about 100 μm—see above for other appropriate ranges). Such incubation periods are also in particular appropriate for a temperature of about 37° C. (e.g. 30-40° C. or 35-45° C.).

In the invention in order for secondary processes to take place, but not primary, droplets may be reduced in size (diameter) by 40-90%, typically 40-70%. Droplets could though be reduced in size by 40-80%, 40-60% or 40-50%. In addition, droplets may be reduced in size by 50-80%, 50-70% or by 50-60%. Droplets could also be reduced in size by 60-90%, 60-80% or 60-70%. In some instances, droplets may be reduced in size by 70-80%, by 70-90% or by 80-90%.

In some instances, the protein is α-synuclein and droplets may be reduced in size (diameter) by 40-70%. Such values are particularly appropriate for average initial diameters in the range of 75-200 μm (optionally 75-150 μm), and α-synuclein seed and monomer concentrations discussed above.

Reductions in droplet size (diameter) may be determined, for example, using microscopy. The droplet arrays may be continuously monitored (imaged), or may be analysed at discrete time points (for example, every 10 minutes or every 30 minutes).

In some instances, alternative techniques may be used to decrease the droplet size (and therefore increase the monomer chemical potential). The example, a high salt-buffer may be flowed in proximity to the to the droplets to allow water to be drawn out by diffusion. Appropriate high-salt buffers would be known to the skilled person and may for example be selected based on characteristics of the droplet (such as the buffer used in the aqueous phase of the droplet). Such buffers could comprise for example NaOH or NaClO₄. Typical reductions in droplet sizes are described above.

Droplets may also simply be reduced in size (diameter) until a signal becomes apparent (for example, a fluorescent signal where a fluorophore is included in the droplets).

Once the droplet size has been reduced to the point where the assessment of aggregation is to be made, conditions with the system may be adjusted to maintain the droplet volume (to maintain the monomer chemical potential) whilst aggregation reactions take place. For example, any heating used to cause evaporation may be removed and the droplets incubated at a temperature where only very minimal evaporation occurs (for example, droplets may be incubated at room temperature). Any flow of high-salt buffer in proximity to the droplets may be ceased.

As mentioned above, in some instances the method further comprises further increasing the monomer chemical potential to a point where primary processes take place and determining again the presence of absence of aggregates within the droplets. The monomer chemical potential may be increased using the methods described above, for example, using evaporation or by passing a high-salt buffer in proximity to the droplets.

In some instances, the monomer chemical potential may be increased by greater than 20 fold or greater than 50 fold (relative to the starting monomer chemical potential in the initial droplets). Typically, the monomer chemical potential is increased by greater than 100 fold relative to the starting monomer chemical potential in the initial droplets. In some instances, monomer chemical potential may be increased by greater than 150 fold, greater than 200 fold, greater than 250 fold, greater than 300 fold or greater than 500 fold (relative to the starting monomer chemical potential in the initial droplets).

With regards to the reduction in droplet size, in order to assess primary processes in some instances the droplet may be reduced in size by at least 70% or at least 80% (relative to the size of the initial droplet at the beginning of the experiment). More typically, the droplet may be reduced in size by at least 90% relative to the size of the initial droplet at the beginning of the experiment. The droplet size may for example be reduced by 70-95%. 70-90% or 70-80% relative to the size of the initial droplet at the beginning of the experiment. The droplet size may also be reduced 80-95%, 80-90% or even 90-95% relative to the size of the initial droplet at the beginning of the experiment.

The required reduction in droplet diameter may in some instances depend on the protein in question. In some circumstances, smaller reductions in diameter may be required, such as 60-95%, 60-90%, 60-80%, 60-70%, or even 50-95%, 50-90%, 50-80%, 50-70% or 50-60%.

As discussed above, once droplet size has been reduced to the point where the assessment of aggregation is to be made, conditions with the system may be adjusted to maintain the droplet size (to maintain the monomer chemical potential) whilst aggregation reactions take place.

### Determining the Presence or Absence of Aggregates Within the Droplets

Once the aggregation reactions have been allowed to occur, the presence or absence of aggregates within the droplets may be determined. In some instances, the presence (or absence) of aggregates is determined by the reagent which is capable of detecting the formation of aggregates (if such a reagent was included in the preparation used to form the droplets). As mentioned above, this reagent may be an aggregation specific fluorophore or an aggregation specific dye. The reagent could also be an antibody which specifically binds protein aggregates, Droplets are typically analysed individually for the presence or absence of aggregates (an individual measurement is taken for each droplet). However, in some cases a global read may also be taken where the overall presence or absence of aggregates is analysed.

When the reagent which is capable of detecting the formation of aggregates is an aggregation specific fluorophore any appropriate means of fluorescence detection may be used. For example, fluorescence may be detected using a spectrofluorometer, fluorescence microscopy or fluorescence scanners (including microplate readers). Similarly, when the reagent which is capable of detecting the formation of aggregates is an aggregation specific non-fuorescent dye (e.g. a beta sheet binding dye—see above), standard microscopy or spectroscopy techniques may be used. A droplet may be identified as positive for aggregation if a fluorescence signal is observed. Fluorescence may be determined at regular intervals, for example by acquiring a number of fluorescence images over time.

Alternatively, in some cases, droplets do not contain a specific reagent which is capable of detecting the formation of aggregates. In these instances, optical microscopy may still be used in order to detect aggregates (for example, bright field microscopy, phase contrast microscopy, light sheet fluorescence microscopy or confocal microscopy). For example, the presence of protein aggregate gels or protein aggregate crystals may be detected using microscopy techniques. Through employing phase contrast microscopy, formation of crystals and fibrillar gels can be detected by the diffraction of light in clear solution vs the surface of the formed structure.

As mentioned above, droplets may be analysed on a static surface, for example where droplets are contained within microwell devices. In other cases, the droplets may be flowed past a detector.

In some instances, methods of the invention may further comprise quantifying the number of protein aggregates capable of seeding further protein aggregation in the original sample by determining the number of droplets positive and negative for aggregation. The number of positive and negative droplets may be counted digitally. The dilution of the original sample containing (or suspected of containing) the aggregates may be factored in order to give an indication of the possible aggregate seed load in the original sample. Where the method is used to detect protein aggregates capable of seeding further protein aggregation in a biological sample from an individual, this may also be used to estimate the aggregate seed load in the individual. In turn, this may be used in order evaluate an individual's prognosis or likelihood of disease progression. The individual may then be treated appropriately in order to try and prevent such disease progression.

### Apparatus

As discussed above, the methods of the invention are typically put into practice using a microfluidics device. Such a device typically comprises means for forming microdroplets and means for storing droplets after formation. Droplets may be arranged into arrays within the device. A storage array may for example be a series of microwells or channels, such as PDMS microwells or channels.

The means for forming droplets is typically coupled to means for storing the droplets e.g. via tubing as mentioned above.

As discussed above, the device may comprise means for mixing the sample comprising or suspected of comprising the aggregate seeds with protein monomers and/or the reagent capable of detecting the presence of aggregates. The reagent may for example be mixed with the protein monomers and then the resulting preparation mixed with the sample comprising or suspected of comprising the aggregate seeds. Such steps may occur inside or outside of the device.

The device may also comprise means for heating the microdroplets.

Microfluidic devices may be fabricated using standard soft-lithography techniques.

### Method of Determining the Presence of Protein Aggregate Seeds by “Shearing”

As discussed above, secondary aggregation processes include the fragmentation of seeds, thus providing additional active sites for monomer binding. This process has been key to the success of current gold-standard seed amplification technologies, including RT-QuIC, yet fragmentation in droplets through their shaking (quaking) is not a viable option. By introducing microbeads typically of a diameter between 0.5-2 μm to the seed and monomer reaction solutions discussed above, droplets containing all three components can be generated. Such droplets can then be injected into a channel narrower than the diameter of the formed droplets, thus allowing the droplets to be compressed, increasing the drag of droplet volume at the channel interface. This increase in drag can induce flow-mediated shear effectively thus cycling the droplet volume. The microbeads encapsulated within the droplet volume can then collide with the seeds, inducing their fragmentation and increase of total aggregate number.

By combining the increase of monomer chemical potentials through droplet evaporation and an increase of fragmentation rate, an amplification of aggregates is obtained in a high-throughput manner.

The invention therefore also provides a method of determining the presence of protein aggregate seeds in a sample by “shearing”. This method comprises (a) providing a preparation that (i) comprises, or is suspected of comprising, protein aggregate seeds of said protein, (ii) comprises monomers of said protein and (iii) comprises an agent capable of fragmenting the aggregate seeds following a collision between the seeds and the agent. Microdroplets are then generated and the shape of the droplets is modified to increase the chance of collisions between the aggregate seeds and the agent capable of fragmenting the aggregate seeds. The presence or absence of aggregation within the droplets is then determined.

The protein may be as described above, and is typically an amyloidogenic protein. Likewise, the aggregate seeds may be as described above and are typically fibrils. The protein aggregate seeds and the protein monomers are derived from the same protein.

As discussed above, the preparation of step (a) may be prepared by mixing the sample comprising or suspected of comprising the aggregate seeds with a different preparation comprising the protein monomers. The sample suspected of comprising the aggregate seeds may be derived from an individual. The individual may be diagnosed with, or suspected of having, a disease associated with protein aggregation. The sample comprising or suspected of comprising the seeds is typically diluted prior to mixing with the monomers.

In addition to the aggregate seeds and monomers, the preparation also comprises as agent capable of fragmenting the aggregate seeds following a collision between the seeds and the agent.

The agent may be microbeads. The microbeads are typically 500 nm-5 μm in diameter, in some instances 1-5 μm in diameter. As above, the microbeads are preferably 0.5-2 μm in diameter. The relatively small diameter of the microbeads allows them to move within the entire volume of the droplet and avoid sedimentation. The microbeads may be present at a concentration of between 0.5 and 2% of the total reaction volume (i.e. of the preparation prior to encapsulation in microdroplets).

The microbeads should be composed of a stiff polymer which will fragment seeds following collision. The microbeads should be made of a material with surface properties that inhibit the binding of protein monomers and inhibit primary nucleation. The microbeads may be polystyrene microbeads.

The agent capable of fragmenting the aggregate seeds following a collision between the seeds and the agent may be added to the preparation comprising the monomers and aggregate seeds. In some instances, the agent capable of fragmenting the aggregate seeds following a collision between the seeds and the agent may be added to either the monomer preparation or to the preparation comprising or suspected of comprising the aggregate seeds. The two preparations are then mixed.

As discussed above, the preparation used to form microdroplets typically comprises a reagent capable of forming the detection of aggregates. This reagent may be as described above. The reagent could be ThT. The reagent may be mixed with the preparation comprising the protein monomers, the aggregate seeds (or suspected aggregate seeds) and the agent capable of fragmenting the aggregate seeds. Alternatively, the reagent may be mixed with any of the individual preparations. In some instances, the reagent may be mixed with the preparation comprising the protein monomers. In other instances, the reagent may be mixed with the preparation comprising the agent capable of fragmenting the aggregate seeds. In further instances, the reagent may be mixed with the sample comprising or suspected of comprising the aggregate seeds. Furthermore the reagent may be mixed with combined preparations of any of the above. All orders of combining the various components of the starting preparation are contemplated.

The concentration of the reagent capable of determining the presence of aggregates is typically as described above. The concentrations of the protein monomers and the aggregate seeds are also typically as described above.

As above, the agent capable of fragmenting the aggregate seeds (e.g. microbeads) may be present at a concentration of between 0.5 and 2% of the total reaction volume i.e. the preparation prior to encapsulation/within the microbeads themselves.

After formation of the starting preparation, the microdroplets may then be formed as described above, in particular using a microfluidics device. The droplet sizes may also be as described above.

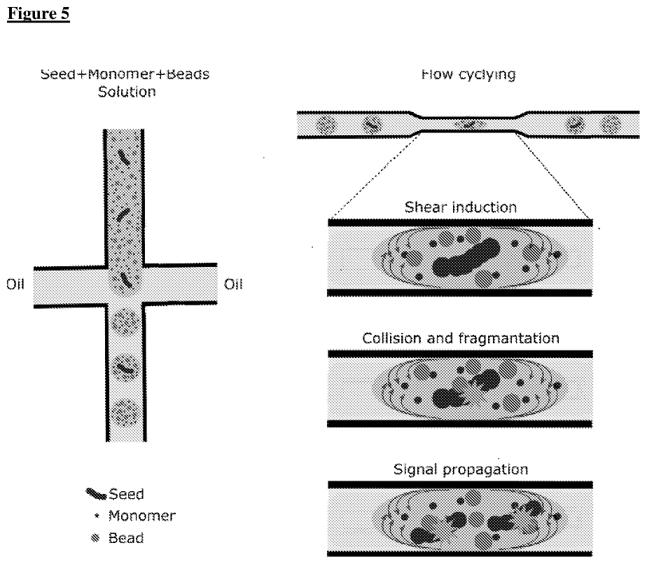

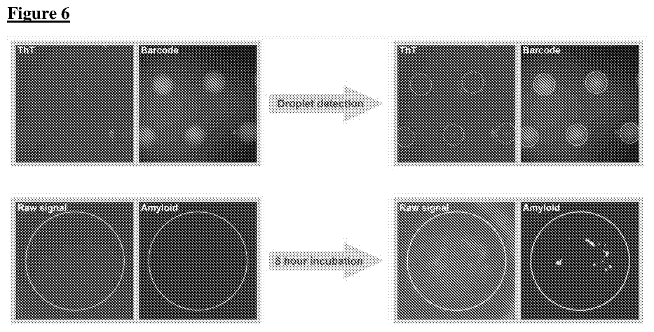

In order to induce fragmentation of the aggregate seeds are then typically flowed from a wider channel into a narrower channel. Flowing of the droplets into the narrower channel compresses and elongates the droplets as shown in FIG. 5. The narrower channel could have, for example, a diameter that is 20-70% smaller than the wider channel. In some instances, the narrower channel could have a diameter that is 20%-50% smaller than the wider channel. In other instances, the narrower channel could have a diameter that is 30-70%, 30-60% or 30-50% smaller than the wider channel. Flowing of the droplets into the narrower channel causes movement of the components within the droplets (stirring within the droplets) and increases collisions of the agent capable of fragmenting the aggregate seeds with the aggregate seeds. Accordingly, the aggregate seeds fragment and then propagate via addition of monomers to form new aggregates. The fragmentation thus contributes to secondary nucleation processes by virtue of increased availability of additional surfaces to which monomers can bind.

The droplets may be flowed through a/the narrower channel a plurality of times (e.g. two or more, five or more, ten or more). The presence or absence of aggregates in the droplets may then be determined as described above.

The apparatus for carrying out this method may also be as described above, except including means for deforming the shape of the droplets (e.g. a section of narrower channel as shown in FIG. 5).

In some instances, the methods of determining the presence of protein aggregate seeds by shearing may be combined with the above described methods of increasing the monomer chemical potential. In others words, evaporation based approaches may be combined with the shearing based approach. Evaporation (increasing the monomer chemical potential) may take place prior to shearing or shearing may take place prior to evaporation. Both approaches may also be combined at the same time (i.e. evaporation may take place at the same time as shearing).

Combining increasing the monomer chemical potential (evaporation) with shearing methods increases the rate of aggregation and propagation of signal.

Whilst the above-described method utilises the addition of agent capable of fragmenting aggregate seeds, in some instances the methods involve modifying the shape of the droplets (as described above) in order to fragment seeds, but without addition of such an agent. These methods may also be combined with the above-described methods of increasing the monomer chemical potential.

### Digital Detection Methods

The methods of the invention may utilise digital detection methods, in particular digital imaging methods, in order to identify the presence of aggregates in droplets. In some instances the methods also involve visualisation of the droplets themselves.

An imaging agent is typically added to the droplets. In some instances, an imaging agent may be added to the droplets, which imaging agent localises to the droplet boundary (for example, the agent localises at the oil-water boundary). The imaging agent may, for example, be a fluorescent dye. Where a reagent capable of detecting the formation of aggregates is included in the preparations/microdroplets, the imaging agent typically provides a signal at a wavelength which is distinct from the wavelength at which the reagent capable of detecting the formation of aggregates provides a signal. In other words, typically the fluorescence of the imaging agent and the reagent capable of detecting the formation of aggregates do not overlap. For example, where the reagent capable of detecting the formation of aggregates is ThT the imaging agent will emit a signal which is distinct from the wavelength at which ThT provides a signal (i.e. the fluorescence of the imaging agent does not overlap with the fluorescence of ThT).

In some instances, the imaging agent could be a fluorescent dye such as Atto 647 or Atto 542.

The imaging agent may be added to the preparations described above which are then formed into microdroplets.

Two imaging channels are then used to image the microdroplets. The first detects at the wavelength of the imaging agent and the second detects at the wavelength of the reagent capable of detecting the formation of aggregates. Imaging at the first wavelength allows the droplet boundary to be determined. This information allows the region of interest to be determined when imaging for formation of aggregates. This step compensates for the fact that a signal from the reagent capable of detecting the formation of aggregates is not typically present in the absence of aggregates, thus making it difficult to determine the location of droplets.

The locations and radii of all droplets are determined via the imaging agent and for each droplet its boundary can be tracked over time by looking for a droplet boundary close to the boundary detected in the previous time frame. Thereby a droplet's history can be traced. The channel for the reagent capable of detecting the formation of aggregates is then used determine the amount of aggregates in each droplet.

The digital imaging method may also comprise (in some instances in addition to the above) means for specifically identifying the position of aggregates. These methods may be applied where a reagent capable of detecting the formation of aggregates is included in the droplet, typically a fluorescent reagent (such as ThT).

Here, the intensity value of each pixel within the droplet is determined. The intensity for an individual pixel is compared with the average pixel intensity for pixels in close proximity to the individual pixel. For example, “close proximity” could may refer to pixels which are within a distance which is comparable to five times a typical aggregate. If the pixel is much brighter than those around it the pixel is labelled as an aggregate signal. This may be determined by the overall signal fluctuation within the droplet. A “signal” may be defined as having more than three standard deviations above the region surrounding the pixel.

The aggregate signal clusters in each droplet can then be calculated by their numbers and sizes. The onset of aggregation can therefore be determined by performing the analysis on all droplets. The lag phase may then be determined from the ensemble measurements.

The following Examples illustrate the invention.

### EXAMPLES

### Materials and Methods

### Expression and Purification of α-Synuclein

Human α-syn WT was expressed and purified using heat treatment, ion exchange and gel filtration chromatography as described previously (Grey et al (2011). Briefly, Escherichia coli BL21 cells overexpressing α-synuclein were collected by centrifugation (20 min, 4000 rpm, 4° C.) in a JLA-8.1000 rotor in a Beckman Avanti J25 centrifuge (Beckman Coulter), resuspended in lysis buffer (10 mM Tris, 1 mM EDTA, protease inhibitor), and lysed by sonication on ice. Following centrifugation (JA-25.5 rotor, 20 min, 18,000 rpm, 4° C.), heat-sensitive proteins were precipitated out of the lysate supernatant by boiling, and subsequently removed by centrifugation (JA-25.5 rotor, 15 min, 18,000 rpm, 4° C.). DNA was precipitated out by incubation with streptomycin sulphate (10 mg/mL, 15 min, 4° C.), and removed by centrifugation. α-synuclein was precipitated out of the supernatant by the slow addition of ammonium sulphate (361 mg/mL) while stirring (30 min, 4° C.). The pellet containing αsynuclein was collected by centrifugation (JA-25.5 rotor, 15 min, 18,000 rpm, 4° C.) and resuspended in 25 mM Tris buffer, pH 7.4, 20° C. Following dialysis to ensure complete buffer exchange, the protein was loaded onto a HiLoad™ 26/10 Q Sepharose high performance column (GE Healthcare), and eluted at ˜350 mM NaCl, 20° C. with a salt gradient from 0 M to 1.5 M NaCl. Selected fractions were subsequently loaded onto a Superdex 75 26/60 (GE Healthcare) at 20° C. and eluted in PBS. pH 7.4, 20° C. Protein concentration was determined by absorbance at 275 nm, using an extinction coefficient of 5600 M⁻¹ cm⁻¹.

### Preparation of α-Syn Seed Fibrils

α-Syn seed fibrils were formed in Eppendorf tubes (Axygen low-bind tubes) at 37° C., with low stirring speed (300 rpm) with a teflon bar and left fibrillating for up to 48 h. Parallel kinetic ThT measurements were performed to assure that the plateau was reached during this time period. Prior to loading into microdroplets, seed fibrils were pre-treated by 1 min continuous sonication at maximum power in a sonicator bath (Struer, Copenhagen, DK) to disperse lumped fibrils. The concentration of seeds is counted as monomer equivalents (Gaspar et al (2017)).

### Seeded Microdroplet Generation

Microfluidic channels were fabricated in polydimethylsiloxane (Dow Corning, Midland, MI, USA) using SU8 on silicon masters and standard soft lithography techniques, and then plasma bonded to glass slides to create sealed single T-junction devices. Following α-Syn seed fibrils sonication, 10-fold sequential dilution seed stock solutions were prepared in both PBS and MES to a concentration of 2 μM-2 pM. Seeds were then further diluted in either PBS or MES in the presence of 25 μM of α-syn WT and 20 or 2 μM of ThT to a final concentration of 200 nM-200 fM. Syringe pumps (neMESYS modules, Cetoni) were used to control the protein solution flow rate of 50 μL/h. Continuous flow of FC-40 oil containing 4% (v/v) fluorosurfactant (RAN Biotechnologies) was introduced to the device at a constant flow rate of 450 μL/h to generate droplets of ˜100 μm diameters.

Droplet Incubation and Imaging Following their generation, droplets were transported to a secondary array device through direct flow in a connecting PTFE tubing. The dual-layered microfluidic device for droplet trapping, analysis, and recovery using droplet buoyancy (Labanieh et al (2015) and Arter et al). In brief, the device utilizes density differences between the continuous and discrete phases to trap floating droplets within thousands of microwells with a diameter of 120 μm in a secondary layer above the main flow stream, allowing for their immobilisation. Following droplet immobilisation, fresh FC-40 solution was injected to remove any droplets not trapped within the microwells. Droplets were then incubated at 37° C. within a heated custom made microscope stage and allowed to evaporate over 24 hours. Continuous imaging of the entire array was achieved using an XY motorised stage (Zaber technologies). ThT fluorescence images were captured using a Niji (Bluebox optics) light source operating at 100% illumination at a wavelength of 445-488 nm, 200 ms exposure time using 10× magnification with 10 min intervals. Image analysis was performed using ImageJ software.

### Results and Discussion

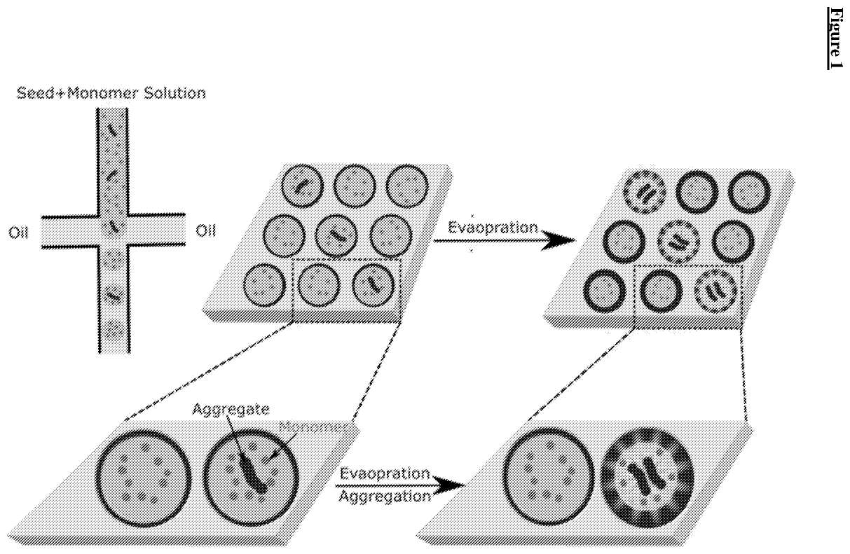

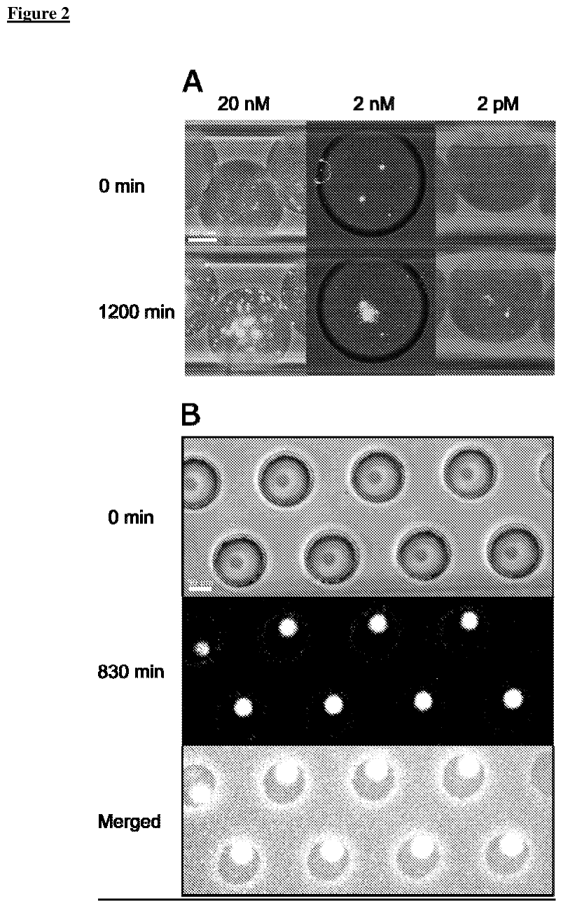

Development was first focussed on the physical platform for the production of parallel microdroplet arrays. Current polydimethylsiloxane (PDMS) based array devices developed through soft lithography techniques were optimised to allow for the formation of a microfluidic device capable of producing and storing thousands of monodispersed droplets, with high trapping efficiency (Arter et al). α-Syn seed fibrils were prepared as described above and then sequentially diluted to concentrations of 2 μM-2 pM. Droplets were then generated by on-chip flowing and mixing solution to be encapsulated, as a dilute solution of α-synuclein protein aggregates at a final concentration of 200 nM-200 fM (monomer equivalent) in the presence of 25 μM α-syn WT monomers and 20 or 2 μM of ThT through their co-flow with the continuous phase composed of fluorinated oil containing 4% fluorosurfactant. By modulating the flow rates and composition of solutions used, a wide range of molecular species can be specifically mixed in solution and immediately encapsulated in droplets of specific volume and incubated to allow the reaction to take place in a controlled manner. Following water-in-oil droplet generation at the junction, droplets were arrayed within a PDMS device and monitored over time (FIG. 1).

Specifically, the intrinsic amplification of aggregates by the autocatalytic self-replication as employed, which means that individual seed aggregates can initiate an aggregation cascade through controlled droplet evaporation and form levels of aggregates detectable macroscopically. By sequential dilution of the aggregate species using the microfluidic approach described above, droplets were formed containing, on average, one or fewer aggregates, along with homogeneous stock solutions of monomers for amplification, to allow for the detection of propagating species in individual droplets.

While the droplet arrays are subjected to controlled volume decrease as PDMS allows for the absorption and evaporation of the aqueous solution contained within (Levin et al (2014)0, the droplets are digitally monitored by bright-field and fluorescent microscopy using specific optical markers, such as ThT, to determine the formation of the self-assembly products and their kinetics under various reaction conditions. As droplet volume decreases over time the chemical potential of the available free monomer increases, leading to secondary nucleation processes contributing to seed amplification and propagation (FIG. 1).

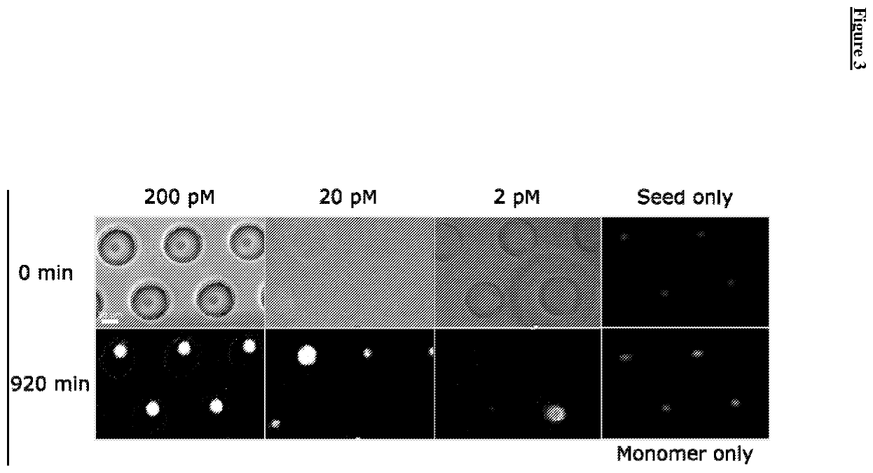

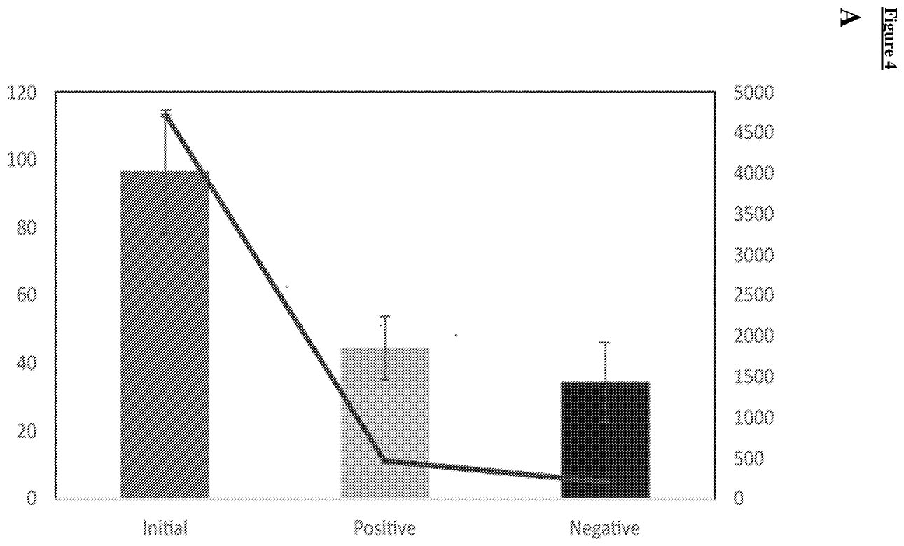

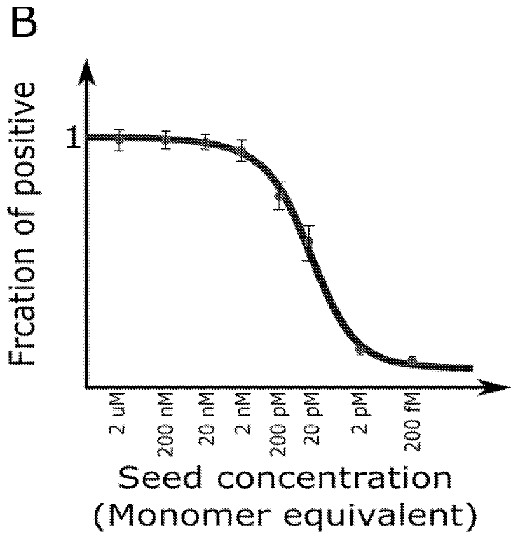

Initially, the detection limit of aggregates through their amplification with and without microdroplet volume decrease was compared. Microdroplets containing seeds at concentrations of 200 nM-200 fM were generated and either trapped within glass capillaries to avoid evaporation, as previously described (Pfammatter et al (2017)), or within PDMS array devices and ThT signal intensity was monitored over time. In the lack of volume reduction found in the sealed glass capillaries, a detection limit of 2 nM was reached over a 1200 min incubation period, as in lower concentrations no significant signal increase was observed (FIG. 2A). However, using the evaporation based approach, a 1000 fold increase in detection limit was observed, where droplets containing 200 pM of seeds could be detected within 830 min, more accurately describing the presence of single aggregates confined within individual microdroplets, rather than their clusters (FIG. 2B). Significantly, while only a minor spatial increase in ThT signal could be observed in capillaries, as droplet volume is decreased while confined within the PDMS device, the entire droplet volume fluoresces, allowing for signal-to-noise enrichment, thus facilitating the quantification of ThT positive droplets.

Absolute quantification of the number of propagation-competent species in the original sample can then be performed by digital counting of the fractions of fluorescence-positive (containing aggregation competent species) and negative droplets for each dilution factor at the assay endpoint (see FIG. 2). An experimental condition screen was further performed to maximise the specific amplification of α-synuclein protein propagon-catalysed reactions, while suppressing spontaneous nucleation of the monomeric substrate protein to minimise false-positives. This stage included systematic screening and optimisation of several experimental parameters, including monomer concentration, temperature, pH-value, buffer and initial droplet volume.

The ability to gain sufficient ThT signal intensity to distinguish between ThT positive and negative droplets was explored, by comparing the signal of droplets following incubation at initial ThT concentration of 20 and 2 μM. It was found that using an initial ThT concentration of 20 μM yields inaccurate positive signal count as with the decrease of microdroplet volume, ThT crashes out of solution and sediments, thus giving a false-positive signal. However, by decreasing the initial ThT concentration to 2 μM, a clear distinction between droplets containing seeds to those devoid of seeds is established.

The ability to accurately quantify ThT positive droplets of the entire droplet milieu was next explored. The diameter of droplets at time 0 and at time of signal appearance was thus compared at several seed concentrations through bright field and fluorescent array imaging acquired every 10 min for a total of 24 hr. Two distinct features were found differentiating ThT positive and negative droplets. First, it was observed that while the initial average diameter of all droplets was 96.6±18 μm. ThT positive droplets began fluorescing at an average diameter of 44.6±9 μm while ThT negative droplets show a slight increase in signal at an average diameter of 34.4±9 μm. Secondly, as signal intensity correlates directly with the system volume and thus with the evaporation rate, ThT positive droplets appear to be fluorescent following a 588 min incubation period, while negative droplets only appear slightly fluorescent following 1200 min period. These results appear consistent within a seed concentration range of 200 nM-200 fM using either PBS or MES based solution. Interestingly, at lower seed concentrations such as 2 pM and 200 fM, only droplets with an initial diameter of 147 eventually displayed a positive signal, while smaller ones appeared as ThT negative. Based on the acquired images it was found that the rate of droplet diameter decrease is 0.9 μm per 10 min. Furthermore, by deriving the volume decrease of droplets over time based on their diameter, it was found that in droplets containing seed a ˜10 fold increase of the free monomer concentration and thus their chemical potential allowed for signal propagation, a ˜22 fold increase in concentration was required for the appearance of signal in negative ones.

Finally, the fraction of ThT positive droplets for each seed concentration was quantified. While such a digital platform allows for the scanning of thousands of droplets containing seed, accurate counting of seed numbers in solution requires the dilution of the sample to allow the encapsulation of single aggregate within individual microdroplets. However, through serial dilution of the sample the fraction of positive signal is dramatically reduced. Thus, while at seed concentration of 2 nM and above close to 100% of all droplets were propagon positive, at concentration of 200 pM, 20 pM, 2 pM and 200 fM the fraction of ThT positive droplets was shown to be reduced. Specifically, at 200 pM seed concentration 70.4% of droplets were propagon positive, while at seed concentrations of 20 and 2 pM, 55.3% and 2.7% of droplets were propagon positive, respectively. Similarly at 2 pM, at 200 fM seed concentration only ˜3% of droplets displayed a positive signal. Thus, it can be concluded that the current detection limit of 2 pM relates to the presence of single seed confined within a single droplet.

To conclude, the development and application of new biophysical techniques to study aberrant protein-protein self-assembly is described, specifically that of α-synuclein that is related to the onset of Parkinson's, on the level of single aggregates. The described platform allows for increased signal-to-noise ratio, permitting the differentiation and direct counting of seeds. Through employing a controlled microdroplet volume reduction, the chemical potential of added monomers is increased, leading to seed amplification throughout the entire droplet volume at lower concentrations and at a shorter time frame then previously described. Thus, using this approach the limit of detection to 10-12 M seed concentration was expanded, while further reducing the experimental time to 12-16 hr. This controlled chemical potential increases the chain reaction leading to the spatial propagation of individual seeds, thus allowing for a direct and precise counting of their presence in solution. While the results presented herein refer to in vitro seed samples, this approach can further enable the development of new diagnostic tools for the presence of seeds in clinical samples, such as CSF and serum, and determine the ability of such seeds to propagate in vivo.

### REFERENCES

Dobson C.M. Trends Biochem. Sci. 24: 329-332 (1999).
Spillantini M.G. Schmidt M.L., Lee V.M., Trojanowski J.Q., Jakes R., Goedert M. Nature 388: 839-840 (1997).
Padrick S.B., Miranker A.D. Biochemistry 41:4694-4703 (2002).
Chiti F., Calamai M., Taddei N., Stefani M., Ramponi G., Dobson C.M. Proc. Natl. Acad. Sci. USA. 4:16419-16426 (2002).
Lotharius J., Brundin P. Nat. Rev. Neurosci. 3:932-942 (2002).
Knowles T.P., Waudby C.A., Devlin G.L., Cohen S.I., Aguzzi A., Vendruscolo M., Terentjev E.M., Welland M.E., Dobson C.M. Science 326:1533-1537 (2009).
Cohen S.I., Linse S., Luheshi L.M., Hellstrand E., White D.A., Rajah L., Otzen D.E., Vendruscolo M., Dobson C.M., Knowles T.P. Proc. Natl. Acad. Sci. USA. 110:975-863 (2013).
Meisl G., Yang X., Hellstrand E., Frohm B., Kirkegaard J.B., Cohen S.I., Dobson C.M., Linse S., Knowles T.P. Proc. Natl. Acad. Sci. USA. 111:938-949 (2014).
Wilham, J. M. et al. Rapid end-point quantitation of prion seeding activity with sensitivity comparable to bioassays. PLoS Pathog. 6, e1001217, 1001217 (2010).
Atarashi, R. et al. Ultrasensitive human prion detection in cerebrospinal fluid by real-time quaking-induced conversion. Nat. Med. 17, 175-178, (2011).
McGuire, L. I. et al. Real time quaking-induced conversion analysis of cerebrospinal fluid in sporadic Creutzfeldt-Jakob disease. Ann. Neurol. 72, 278-285, (2012).
Shahnawaz, M. et al. Development of a Biochemical Diagnosis of Parkinson Disease by Detection of alpha-Synuclein Misfolded Aggregates in Cerebrospinal Fluid. JAMA Neurol. 74, 163-172, (2017).
Fairfoul, G. et al. Alpha-synuclein RT-QuIC in the CSF of patients with alpha-synucleinopathies. Ann. Clin. Transl. Neurol. 3, 812-818, (2016).
Groveman, B. R. et al. Extended and direct evaluation of RT-QuIC assays for Creutzfeldt-Jakob disease diagnosis. Ann. Clin. Transl. Neurol. 4, 139-144, (2017).

Saijo, E. et al. Ultrasensitive and selective detection of 3-repeat tau seeding activity in Pick disease brain and cerebrospinal fluid. Acta Neuropathol. 133, 751-765, (2017).
Salvadores, N., Shahnawaz, M., Scarpini, E., Tagliavini, F. & Soto, C. Detection of misfolded Abeta oligomers for sensitive biochemical diagnosis of Alzheimer's disease. Cell Rep 7. 261-268, (2014).
Saijo, E. et al. Ultrasensitive RT-QuIC seed amplification assays for disease-associated Tau, α-synuclein, and prion aggregates. Methods Mol. Biol. 1873, 19-37 (2019).
Han, J.-Y. et al. RT-QuIC-based detection of alpha-synuclein seeding activity in brains of dementia with Lewy Body patients and of a transgenic mouse model of synucleinopathy. Prion 14, 88-94 (2020).
Pfammatter, M. et al. Absolute Quantification of Amyloid Propagons by Digital Microfluidics. Anal. Chem. 89, 12306-12313, (2017).
Kopp, M.R.G. et al. Microfluidic shrinking droplet concentrator for analyte detection and phase separation of protein solutions. Anal. Chem. 92, 5803-5812 (2020).
Levin A. et al. Self-assembly-mediated release of peptide nanoparticles through jets across microdroplet interfaces. ACS Appl. Mater. Interfaces, 10, 27578-27583 (2018).
Grey, M., Linse, S., Nilsson, H., Brundin. & Sparr, E. (2011). Membrane interaction of α-synuclein in different aggregation states. J. Parkinson's Disease I, 359-371.
Gaspar R. et al. Secondary nucleation of monomers on fibril surface dominates α-synuclein aggregation and provides autocatalytic amyloid amplification. Q. Rev. Biophys. 50, e6 (2017).
Labanich, L., Nguyen, T.N., Zhao, W. and Kang D.-K. Floating droplet array: an ultrahigh-throughput device for droplet trapping, real-time analysis and recovery. Micromachines (Basel). 6, 1469-1482 (2015).
Arter. W.E., et al. Rapid generation of protein condensate phase diagrams using combinatorial droplet microfluidics. bioRxiv doi: https://doi.org/10.1101/2020.06.04.132308.
Levin A. et al. Ostwald's rule of stages governs structural transitions and morphology of dipeptide supramolecular polymers. Nat. Comm. 5, 1-8 (2014).
Meisl G., Yang X., Hellstrand E., Frohm B., Kirkegaard J.B., Cohen S.I., Dobson C.M., Linse S., Knowles T.P. Proc. Natl. Acad. Sci. USA. 111:9384-9389 (2014).
Wagner. O et al Biocompatible fluorinated polyglycerols for droplet microfluidics as an alternative to PEG-based copolymer surfactants Lab Chip 16, 65-69 (2016)
Törnquist M et al Secondary nucleation in amyloid formation Chem. Commun. 54, 8667-8684 (2018)

1. A method of detecting aggregates of a protein that are capable of seeding further protein aggregation, said method comprising:
(a) providing a preparation that (i) comprises, or is suspected of comprising, protein aggregate seeds of said protein and (ii) comprises monomers of said protein;
(b) generating microdroplets of the preparation of (a);
(b) increasing the monomer chemical potential within the droplets to a point where secondary aggregation processes take place, but not primary aggregation processes; and
(c) determining the presence or absence of aggregation within the droplets;
wherein the concentration of protein aggregate seed in the mixture of step (a) is such that the overwhelming majority of microdroplets of step (b) will each contain either one or zero seeds.

2. The method of claim 1, wherein the method further comprises a step of mixing a sample that comprises, or is suspected of comprising, the protein aggregate seeds with the monomers to form the preparation of step (a).

3. The method of claim 1 or 2, wherein the preparation of (a) further comprises a reagent which is capable of detecting the formation of aggregates, and step (c) comprises detecting aggregation via said reagent.

4. The method of claim 3, which further comprises a step of mixing a sample that comprises, or is suspected of comprising, the protein aggregate seeds with the monomers and the reagent which is capable of detecting the formation of aggregates to form the preparation of step (a).

5. The method of any one of the preceding claims, wherein the concentration of seeds in the preparation of (a) is between 20 fM and 2 nM monomer equivalent, optionally 200 fM and 2 nM monomer equivalent, further optionally between 2 pM and 2 nM monomer equivalent, or between 2 pM and 1 nM monomer equivalent.

6. The method of any one of the preceding claims, wherein the concentration of monomers in the preparation of (a) is 1-100 μM, optionally 10-50 μM, 20-30 μM or about 25 μM.

7. The method of any one of the preceding claims, wherein the protein is an aggregation-prone protein.

8. The method of claim 7, wherein the aggregation-prone protein is amyloidogenic.

9. The method of claim 8, wherein the amyloidogenic protein is selected from Aβ42, α-synuclein, tau, huntingtin, atrophin-1, ataxin (1,2,3,6,7, 8 12,17), amylin, prion protein, (pro)calcitonin, atrial natriuretic factor, apoliprotein AI, apoliprotein AII, apoliprotein AIV, serum amyloid, medin, (apo) serum AA, prolactin, transthyretin, lysozyme, β-2 microglobulin, fibrinogen α chain, gelsolin, keratopthelin, β-amyloid, cystatin, ABriPP immunoglobulin light chain AL, immunoglobulin heavy chain, S-IBM, islet amyloid polypeptide, insulin, lactadherin, lactoferrin, tbn, leukocyte chemotactic factor-2, AbriPP, ADanPP, lung surfactant protein, galectin 7, corneodesmosin, lactadherin, kerato-epithelium, odontogenic ameloblast-associated protein, semenogelin 1 and enfurvitide.

10. The method of claim 9, wherein the protein is α-synuclein.

11. The method of claim 3, wherein the reagent which is capable of detecting the formation of aggregates is a an aggregation specific fluorophore.

12. The method of claim 11, wherein the reagent comprises thioflavin T, thioflavin S or thioflavin X.

13. The method of claim 12, wherein the reagent comprises thioflavin T and the thioflavin T is present at a concentration of between 500 nM and 15 μM, optionally between 1 μM and 10 μM, further optionally between 1 and 5 μM or at a concentration of about 2 μM.

14. The method of claim 3, wherein the reagent that is capable of detecting the formation of aggregates is an aggregation specific non-fluorescent dye.

15. The method of claim 14, wherein the reagent comprises Congo red.

16. The method of any one of claims 1-10, wherein the presence of absence of aggregates within the droplets is determined by bright field microscopy, phase contrast microscopy, light sheet fluorescence microscopy of confocal microscopy.

17. The method of any one of the preceding claims, wherein dilution of the sample comprising or suspected of comprising the seeds is used to prepare the mixture of step (a).

18. The method of any one of the preceding claims, wherein the monomer chemical potential is increased 5 to 100 fold, optionally 5 to 50 fold or 5 to 20 fold, further optionally wherein the protein is α-synuclein and the monomer chemical potential is increased 5 to 20 fold.

19. The method of any one of the preceding claims, wherein the monomer chemical potential is increased by evaporation of the droplets.

20. The method of any one of claims 1 to 18, wherein the monomer chemical potential is increased by flowing a high-salt buffer in proximity to the droplets.

21. The method of claim 19 or 20, wherein the average droplet diameter is reduced by 40 to 70%.

22. The method of any one of the preceding claims, wherein the initial average droplet diameter is 75 to 200 μm, optionally about 100 μm.

23. The method of any one of the preceding claims, which further comprises modifying the shape of the microdroplets.

24. The method of claim 23, which comprises flowing the droplets from a wider channel into a narrower channel in order to compress the droplets.

25. The method of claim 24, wherein the diameter of the narrower channel is 20-70% smaller than the diameter of the wider channel, optionally 30-50% smaller.

26. The method of any one of the preceding claims, which further comprises including an agent capable of fragmenting aggregate seeds following a collision between the seeds and the agent in the preparation of step (a) and modifying the shape of the microdroplets to increase the chance of collisions between the aggregate seeds and the agent capable of fragmenting the seeds.

27. The method of claim 26, wherein the method comprises flowing the droplets from a wider channel into a narrower channel in order to compress the droplets.

28. The method of claim 27, wherein the diameter of the narrower channel is 20-70% smaller than the diameter of the wider channel, optionally 30-50% smaller.

29. The method of claim 26, 27 or 28, wherein the agent capable of fragmenting aggregate seeds is microbeads.

30. The method of claim 29, wherein the microbeads have a diameter of 500 nm-5 μm, optionally 1 μm-5 μm.

31. The method of claim 29 or 30, wherein the microbeads are present at concentration of between 0.5 and 2% of the total reaction volume.

32. The method of any one of the preceding claims, wherein the method further comprises further increasing the monomer chemical potential to a point where primary processes take place and determining again the presence or absence of aggregation within the droplets.

33. The method of claim 32, wherein the monomer chemical potential is increased by greater than 20 fold, greater than 50 fold or greater than 100 fold, optionally wherein the protein is α-synuclein and the monomer chemical potential is increased by greater than 20 fold.

34. The method of any one of the preceding claims, wherein individual droplets are analysed on a static surface.

35. The method of any one of claims 1-33, wherein droplets are flowed past a detector.

36. The method of any one of the preceding claims, wherein the protein is α-synuclein and wherein:
(a) the concentration of seed in the preparation of (a) is between 200 fM and 2 nM monomer equivalent, optionally between 2 pM and 2 nM monomer equivalent, or between 2 pM and 1 nM monomer equivalent;
(b) the concentration of protein monomer in the preparation of (a) is about 25 μM;
(c) the preparation of (a) comprises about 2 μM thiovlain T;
(d) the buffer for the preparation of (a) is PBS or MES;
(e) the monomer chemical potential is increased by evaporation of the droplets, optionally wherein evaporation is conducted at about 37° C. for between 9 and 16 hours; and
(f) the initial average droplet diameter is 75 to 200 μM.

37. The method of claim 36, wherein the preparation of (a) further comprises microbeads at a concentration of between 0.5 and 2% of the total reaction volume.

38. The method of claim 36 or 37, wherein the monomer chemical potential is increased by 5 to 20 fold and/or the average droplet diameter is reduced by 40 to 70% in order for secondary process to take place.

39. The method of any one of the preceding claims, wherein the method further comprises quantifying the number of protein aggregates capable of seeding further protein aggregation in the original sample by determining the number of droplets positive and negative for aggregation.

40. The method of any one of the preceding claims, wherein the method is used to detect protein aggregates capable of seeding further protein aggregation in a biological sample from an individual.

41. The method of any one of the preceding claims, wherein at least 90% of the microdroplets contain either one or zero aggregate seeds.

42. A method of detecting aggregates of a protein that are capable of seeding further protein aggregation, said method comprising:
(a) providing a preparation that (i) comprises, or is suspected of comprising, protein aggregate seeds of said protein and (ii) comprises monomers of said protein;
(b) generating microdroplets of the preparation of (a);
(c) modifying the shape of the droplets;
(d) determining the presence or absence of aggregation within the droplets;
wherein the concentration of protein aggregate seed in the mixture of step (a) is such that the overwhelming majority of microdroplets of step (b) will each contain either one or zero seeds.

43. The method of claim 42, wherein step (c) comprises flowing the droplets from a wider channel into a narrower channel in order to compress the droplets.

44. The method of claim 43, wherein the diameter of the narrower channel is 20-70% smaller than the diameter of the wider channel, optionally 30-50% smaller

45. The method of claim 42, wherein the preparation of (a) further comprises an agent capable of fragmenting the aggregate seeds following a collision between the seeds and the agent.

46. The method of claim 45, wherein the method further comprises a step of mixing a sample that comprises, or is suspected of comprising, the protein aggregate seeds with the monomers and the agent capable of fragmenting the aggregate seeds to form the preparation of step (a).

47. The method of any one of claims 42-46, wherein the preparation of (a) further comprises a reagent which is capable of detecting the formation of aggregates, and step (d) comprises detecting aggregation via said reagent.

48. The method of any one of claims 42-47, wherein the concentration of seeds in the preparation of (a) is between 20 fM and 2 nM monomer equivalent, optionally 200 fM and 2 nM monomer equivalent, further optionally between 2 pM and 2 nM monomer equivalent, or between 2 pM and 1 nM monomer equivalent.

49. The method of any one of claims 42-48, wherein the concentration of monomers in the preparation of (a) is 1-100 μM, optionally 10-50 μM, 20-30 μM or about 25 μM.

50. The method of any one of claims 42-49, wherein the protein is as defined in any one of claims 7-10.

51. The method of claim 47 wherein the reagent which is capable of detecting the formation of aggregates is as defined in any one of claims 11-15.

52. The method of any one of claims 42-50, wherein the presence of absence of aggregates within the droplets is determined by bright field microscopy, phase contrast microscopy, light sheet fluorescence microscopy of confocal microscopy.

53. The method of any one of claims 42-52, wherein dilution of the sample comprising or suspected of comprising the seeds is used to prepare the mixture of step (a).

54. The method of any one of claims 45-53, wherein step (c) comprises flowing the droplets from a wider channel into a narrower channel in order to compress the droplets.

55. The method of claim 54, wherein the diameter of the narrower channel is 20-70% smaller than the diameter of the wider channel, optionally 30-50% smaller.

56. The method of any one of claims 45-55, wherein the agent capable of fragmenting the aggregate seeds is microbeads.

57. The method of claim 56, wherein the microbeads are between 500 nm and 5 μm in diameter, optionally between 1 μm and 5 μm in diameter.

58. The method of any one of claims 42-57, wherein the droplets are flowed from the wider channel into the narrower channel a plurality of times.

59. The method of any one of claims 42-57, wherein individual droplets are analysed on a static surface or wherein the droplets are flowed past a detector.

60. The method of any one of claims 42-59, wherein the method further comprises quantifying the number of protein aggregates capable of seeding further protein aggregation in the original sample by determining the number of droplets positive and negative for aggregation.

61. The method of any one of claims 42-60, wherein the method is used to detect protein aggregates capable of seeding further protein aggregation in a biological sample from an individual.

62. The method of any one of claims 42-61, wherein at least 90% of the microdroplets contain either one or zero aggregate seeds.

63. The method of any one of the preceding claims, wherein the presence of aggregates is determined using a digital imaging detection method.

64. The method of claim 63, which comprises identifying the pixel intensity values for each pixel in the droplet and comparing the intensity value for a selected pixel to the average pixel intensity for pixels in close proximity to the selected pixel.

65. The method of claim 64, wherein a pixel is labelled as providing an aggregate signal if the pixel is brighter than those in the surrounding area.

66. The method of any one of the preceding claims, which further comprises adding an imaging agent to the microdroplets in order to identify the droplet boundaries.

67. The method of claim 66, wherein the imaging agent is a fluorescent dye.

68. The method of claim 66 or 67, wherein the microdroplets comprise a preparation comprising a reagent which is capable of detecting the formation of aggregates and the imaging agent and the reagent which is capable of detecting the formation of aggregates may be visualised at different wavelengths.
